(19) United States
(12) Patent Application Publication
(10) Pub. No.: US 2015/0343166 A1
(43) Pub. Date: Dec. 3, 2015

(54) APPARATUS TO DRY, WEIGH AND SANITIZE BODY

(71) Applicants: Jason RYU, TUSTIN, CA (US); Joon CHOI, PALISADES PARK, NJ (US); Paul RYU, ENGLEWOOD CLIFFS, NJ (US)

(72) Inventors: Jason RYU, TUSTIN, CA (US); Joon CHOI, PALISADES PARK, NJ (US); Paul RYU, ENGLEWOOD CLIFFS, NJ (US)

(21) Appl. No.: 14/418,735

(22) PCT Filed: Aug. 17, 2012
(86) PCT No.: PCT/US2012/051286

§ 371 Date: Aug. 18, 2015

Related U.S. Application Data
(60) Provisional application No. 61/677,805, filed on Jul. 31, 2012.

(51) Int. Cl. A61M 16/10; A47K 17/02; G01G 19/44; A47K 10/48

(52) U.S. Cl. CPC A61M 16/10; A47K 10/48; A47K 17/02; G01G 19/44

(57) ABSTRACT

A full body drying system is an apparatus that can dry a full body with forced air, measure the weight of the body, and provide other health enhancing functions. The apparatus comprises a lower frame, and an upper frame, a blower unit, a foot plate, and a weighing scale. The lower frame and the upper frame provide the other components of the apparatus with the proper structural support. Once the user steps onto the foot plate, the blower unit provides the motorized forced air carrying massive amount of anions, which is used to dry and also to invigorate the body, and simultaneously the body weight is measured and displayed. In addition, around the user's feet area, UV light combined with antimicrobial plastic material used for the foot plate provide effective disinfection of the user's feet, which is further enhanced by the disinfecting ability of anions in the forced air stream.

### FIELD OF THE INVENTION

The present invention relates generally to the field of personal care apparatus. More particularly, this invention is in the technical field of forced air body drying apparatus. More specifically, this invention is a forced air body drying apparatus integrated with body weighing scale, and is equipped with health enhancing devices of negative ion generators and sanitizing UV lighting fixtures along with use of antimicrobial material.

### DESCRIPTION OF THE RELATED ART

There are known forced air body drying systems for use after shower. Most of them are large systems built in or fixed to shower stall structures, or even if movable, requiring quite bulky set-up. Small drying apparatuses are mostly for limited drying of hands or feet.

There are many different types of commercially available body weighing scale modules with digital display to be adapted to this invention as a component.

Negative ions or anions have the effects of purifying blood, activating cells, increasing immunity, controlling the autonomic nervous system removing toxic materials generated by electromagnetic waves, eliminating dust and bacteria, adjusting humidity and removing odor. When a person inhales such anions from air, there occur several effects of promoting the metabolism of cells, purifying blood, tranquilizing nerves, relieving fatigue and promoting appetite. And in recent years, anion has been drawing attention from the medical field of environmental therapy as well.

Under natural conditions, it is generally known that the number of anion count per 1 cc in fresh country air runs 2,000-4,000 and the number rises sharply many-folds to tens of thousands in wide variance in forests, hot springs, water-falls and a seashore area, etc.

There are various methods known to artificially generate these anions indoors, and in recent years such anion generators are incorporated into many devices and systems. This invention, however, calls for a specific type that generates anions by charged metal fiber brush tip as further described below for the reason that this particular method of anion generation produces negligible amount of ozone which is usual byproduct of anion generation.

In addition, it is known that UV light is an effective disinfectant, and considered to be a good cure for eczema and athlete's foot, and there are certain plastics material known to be antimicrobial through use of natural and harmless substance named Phytoncides which are antimicrobial allelochemic volatile organic compounds derived from plants, thus simultaneous combination of anion and UV light and use of aforesaid antimicrobial plastics would afford a very effective disinfection.

### BACKGROUND OF THE INVENTION

Current methods of drying an individual and body dryers are ineffective in the overall cleaning of the individual. Towels collect vast amounts of dirt and over time lose their effectiveness as a cleaning apparatus. Additionally, current body drying apparatuses are large and cumbersome, making them near impossible to use.

Negative ions or anions have the effects of purifying blood, activating cells, increasing immunity, controlling the autonomic nervous system removing toxic materials generated by electromagnetic waves, eliminating dust and bacteria, adjusting humidity and removing odor. When a person inhales such anions from air, there occur several effects of promoting the metabolism of cells, purifying blood, tranquilizing nerves, relieving fatigue and promoting appetite. And in recent years, anion has been drawing attention from the medical field of environmental therapy as well.

It is therefore an object of the present invention to provide an apparatus that is a forced air body drying apparatus integrated with body weighing scale, and is equipped with health enhancing devices of negative ion generators and sanitizing UV lighting fixtures along with use of antimicrobial material.

### SUMMARY OF THE INVENTION

The objective of this invention is to provide a potable and easy to use apparatus for everyday personal care needs that combines three different but simultaneous functions that can be turned on in one simple action of stepping on the apparatus affording: thorough body drying by forced air stream, checking body weight, while breathing anions and exposing entire body surface to anion bombardment, thereby achieving health benefits of anions, and also, as an optional addition, shedding sanitizing UV light over toes and the crevices in between, thereby further sanitize toe area which is most vulnerable to eczema and athlete's foot.

In general, after taking shower or bath, whether at home or at work or at a public place such as sports center, health club, etc., the residual water on body is wiped with towel waiting for natural drying up of body moisture often letting residual moisture stay for a while particularly in crevices of the lower body resulting in potential health hazards such as eczema, athlete's foot, frostbite, etc. Currently motorized forced air dryers are available only for hand or foot drying on rather limited function and usage, not to mention whole body drying.

It is a common habit of most people particularly those who are conscious of health and beauty to check their body weight after a bath or shower by stepping on a body scale of any sort.

In the first embodiment, there is the main air chamber into which forced air stream from the blower is drawn building up air pressure to be released through multiple outlets placed in specific arrangement and to pass through the nozzles above the chamber, and discharged through induction holes that are arranged for optimum distribution of the upward forced air stream around feet. At the center of the main air chamber, there is a partitioned and isolated space which reduces the volume of the chamber thereby helping air pressure build-up, and guide and separate the incoming air stream evenly toward each foot area. Attached to the main air chamber is the air blower unit that is turned on by the signal from the weight scale at the bottom of the apparatus upon a user stepping on the apparatus. The air stream blown out of induction holes flowing upward around the feet of the person standing on the apparatus in bare body will follow the contour of the body surface by coanda effect sweeping away the residual moisture on body surface.

In another embodiment, there are heating coils inside the air passage from the air blower to the main air chamber to warm up the air if the heater is turned on as an option by a separate select switch on outside the apparatus, allowing the user choice or warm or cool air.

In another embodiment, inside the isolated center space in the main air chamber, there are negative ion generators from which metal fiber brushes are pulled through the holes directly facing against in the air blower outlet so that the anions released from the metal fiber brushes are carried away by the air stream. As the air stream flow upward sweeping the user's body surface, anions in the air stream will be in contact with the skin while dispersing into the ambient air the user breathes.

In another embodiment, at the bottom of the apparatus, there is a weight scale unit whose weighing top plate is attached to the bottom of the rest of the apparatus, and the scale unit is also the base of the apparatus. When the scale is turned on by a user stepping on the apparatus, signal for the measured weight will be sent to the digital display unit installed at the top of the apparatus, while also turning on the air blower.

In another embodiment, at the upper part of the apparatus right in front and above the area for toes, there is a UV sterilizing light fixture (optional item) with reflector so as to further strengthen sterilization of toes and particularly the crevices between toes that are most prone to eczema or athlete's foot, in addition to the sanitization by anions.

### BRIEF DESCRIPTION OF THE DRAWINGS

FIG. 1 is a top-side perspective illustration of the present invention.

FIG. 2 is an exploded illustration of components of the present invention.

FIG. 3 is a top side illustration of a user's feet positions when standing on the present invention.

FIG. 4 is a right-side cross-sectional view illustration showing shapes and locations of the components of the present invention.

FIG. 5 is a top-side illustration of initial air flow inside the main air chamber of the present invention.

FIG. 6 is a top-side illustration of details of the configuration and relative sizes of the induction holes of the present invention.

FIG. 7 is a front-side illustration of details of the nozzles and the direction of air stream dispersion of the present invention.

FIG. 8 is a front-side illustration of the variance of the induction hole shape for guiding the air stream near the foot side surface and the resultant direction of the air flow of the present invention.

FIG. 9 is a right-side illustration of the user standing on the apparatus and the flow direction of the upward air stream near the body thoroughly sweeping through the body surface contour influenced by coanda effect in the present invention.

FIG. 10 is a front-side illustration of a typical unit of negative ion generator with an embedded power supply eczema or athlete's foot, in addition to the sterilization by anions in the present invention.

### DETAIL DESCRIPTIONS OF THE INVENTION

All illustrations of the drawings are for the purpose of describing selected versions of the present invention and are not intended to limit the scope of the present invention.

Referring now to the invention in more detail referenced to the figures attached, one example of execution of this invention as a practical body dryer-scale apparatus is described herein, though the shapes and specific arrangement of the components described herein should not be construed as limitations to the execution of this invention. Rather, there should be many variations of the shapes and component arrangement without diminishing the benefits of this invention.

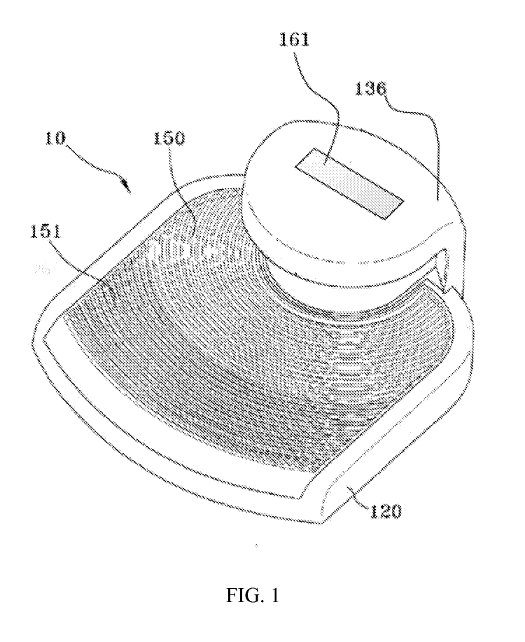

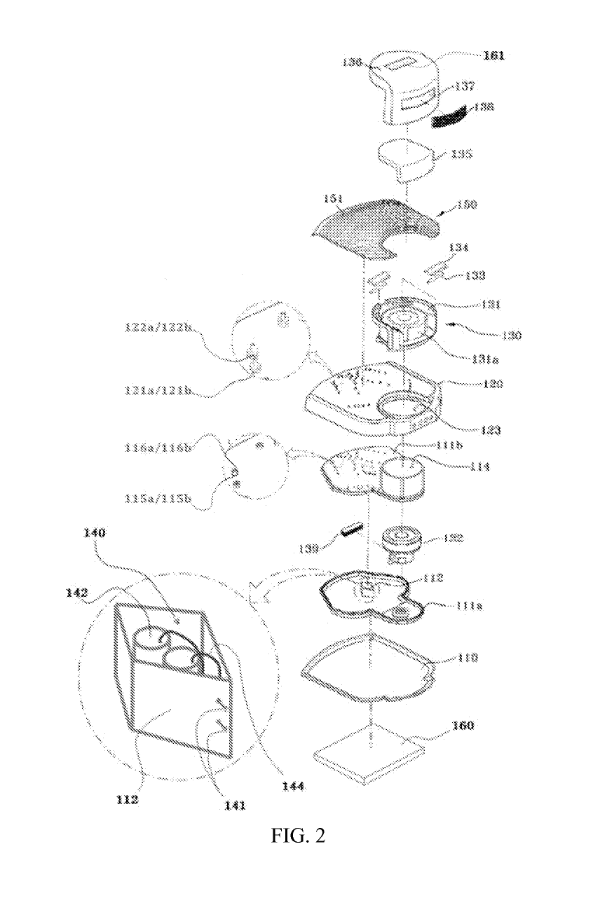

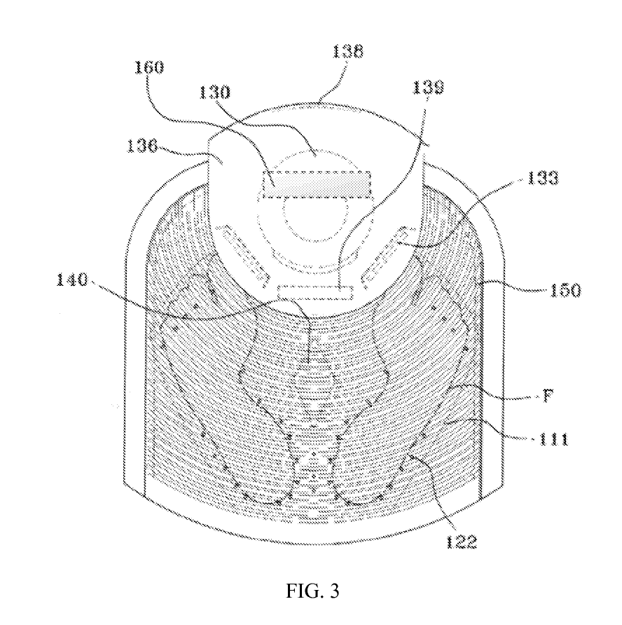

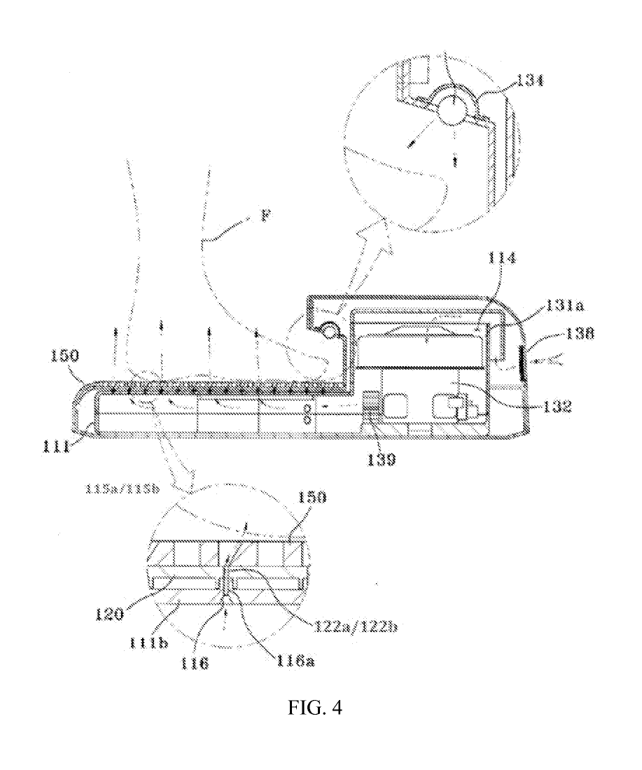

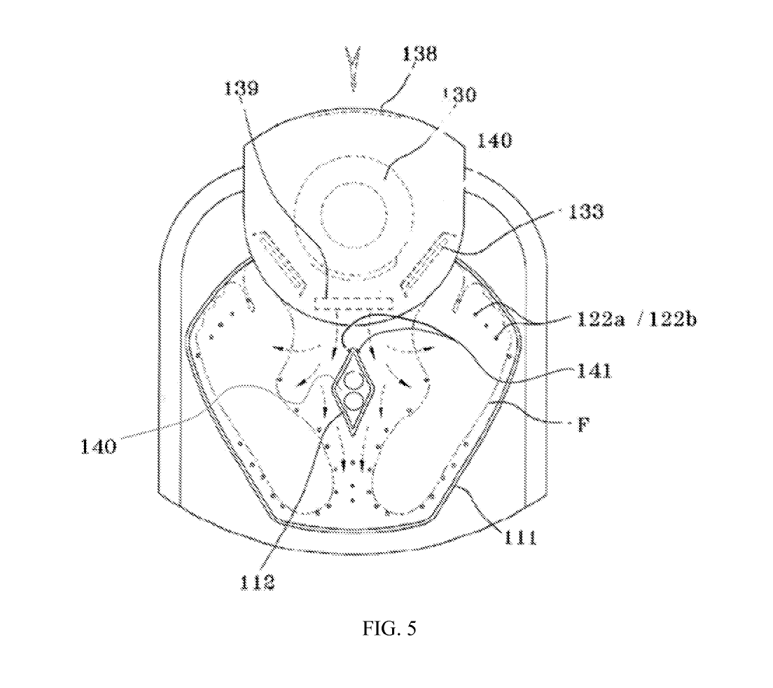

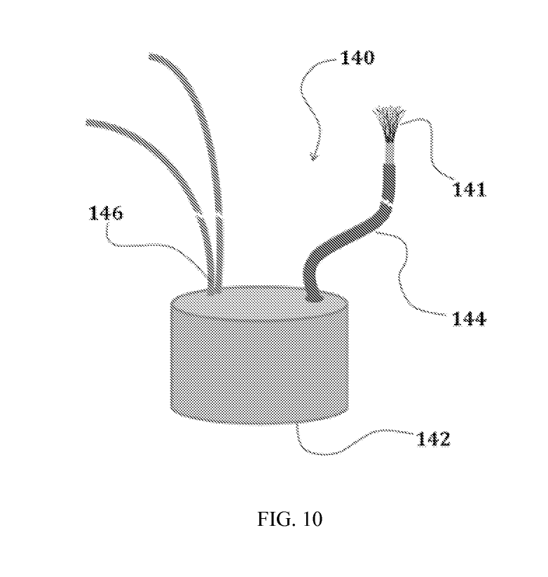

In FIG. 1, the apparatus 10 is shown in perspective view, and in FIG. 2, all components of the apparatus 10 is shown and identified in an exploded skew drawing, and in FIG. 3, the apparatus 10 is shown in ichnography depicting relative locations of the components and the air outlet nozzles 122, and FIG. 4 through FIG. 9 depict path and direction of air stream at different components and parts of the apparatus in operation, and FIG. 10 shows an example of an anion generator 140 in perspective view.

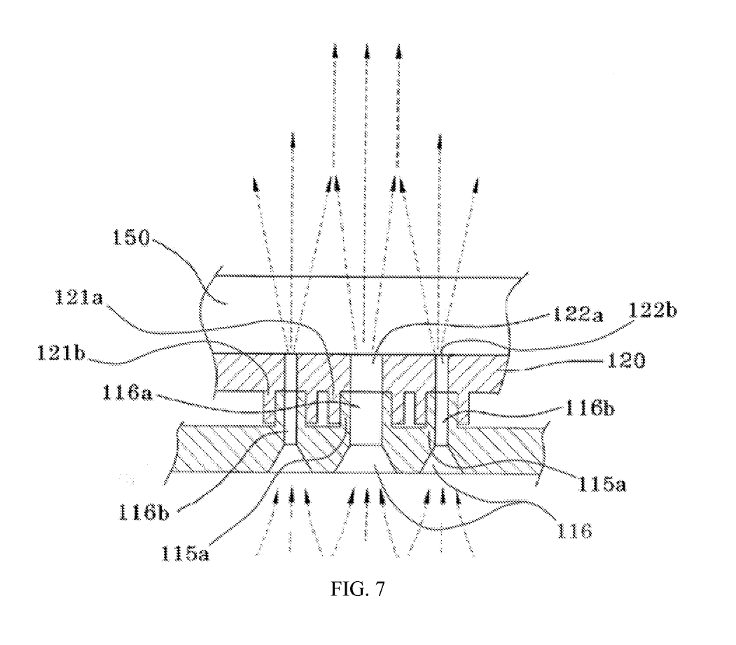

Referring to FIG. 1 and FIG. 2, the apparatus 10 comprises lower frame 110, upper frame 120, blower unit 130 and the foot plate 150. At the top face of the cover of blower unit 130 is a digital weight display window 161 shown. Sits on lower frame is the air chamber 111 formed by lower air chamber part 111a with the air flow guide partition 112 attached at the center, and the upper air chamber part 111b that joins with the lower air chamber part 111a and has on its upper side a number of large and small joining nipples 115a and 115b forming air outlets of large sizes 116a and small sizes 116b and toward its backside attached upward a cylindrical air inlet opening 114 through which outside air is drawn into the air chamber 111. The air outlets 116a and 116b have air entry openings 116 (as shown in FIG. 7) much larger than the sizes of the air outlet 116a or 116b into which pressurized air in the air chamber 111 is pushed into, and the tapered passage from the entry opening to the much smaller outlets 116a or 116b further increases the pressure of the air passing through.

And at the back side of the upper frame 120 is the passage opening 123 through which cylindrical air inlet opening 114 passes standing above the upper frame 120, and throughout the upper face of the upper frame 120, there are large and small joining receptacles 121a and 121b extruding downward at the spots matching with the joining nipples 115a and 115b (of the lower air chamber part 111a) so as to line up the matching size holes to air outlets 116a and 116b, thereby together forming large and small nozzles 122a and 122b for discharge of air from the air chamber 111.

The locations of these nozzles 122a and 122b are configured to surround bottom of the user's feet F particularly around the hill area and to be directly underneath the crevices between the toes, with the naturally standing user's feet F to be positioned on both sides of the air flow guide partition 112.

The blower unit 130 comprises a blower housing 131 where an air blower fan 132 is installed which draws air from outside and blow into the air chamber 111 through the air passage 131a then fan cover 135 that naturally directs the air into the air chamber 111.

Also installed in the blower housing 131 is an air heater 139 that heats the air blown into the air chamber for the apparatus to discharge warm air toward the user, if so selected. A honeycomb type PTC ceramic heater is preferred in this invention so as to heat the air as the air pass through the honeycomb.

At the top of the upper frame 120 is the blower unit cover 136 with air filter 138 at its backside, which would be a common HEPA filter.

Since all electrical components of this invention are common electrical items of known control circuitry, control circuitry of this invention is omitted herein.

Referring to FIG. 2, assembly of this invention starts with fixing the blower fan 132 at its position on the lower air chamber part 111a, insert upper part of the blower fan 132 through the cylindrical air inlet opening 114 of the upper air chamber part 111b, thereby join the two air chamber parts 111a and 111b to form the air chamber 111, which then sits on the lower frame 110, and the weighing scale unit 160 is attached underneath the lower frame 110, then the upper frame 120 will be joined with the lower frame 110, matching the joining nipples 115a and 115b of the air chamber 111 with the joining receptacles 121a and 121b thereby aligning the nozzles 122a and 122b and let the cylindrical air inlet opening 114 sticks out through the passage opening 123.

Next, join the blower housing 131 to the passage opening 123 such that the blower fan 132 fits to the air passage 131a, and at the front top of the blower housing 131, there are UV lamp fixtures 133 with reflectors 134 installed such that the UV light sheds directly upon toe area.

Next, join the blower fan cover 135 to the blower housing 131 cautioning that the blower fan cover 135 would not interfere with the air passage 131a, then join the blower unit cover 136 with air filter 138 to the upper frame 120 over the blower unit 130.

The weighing scale 160 is a type that comprises a load sensor which, upon a user standing on the apparatus 10, sends signal to the digital display 161 at the top of the blower cover unit 136 displaying the measured weight and to the main power switch of the apparatus 10 turning on the blower fan 132 and the UV lamps 133 and the anion generation unit 140 to discharge anions from the metal fiber brushes 141 sticking out through the end side of the air flow guide partition 112 with the tips of metal fiber brush 141 facing directly against the incoming air stream from the blower fan 132. All of such discharged anions will be carried away in the air stream.

Lastly, the foot plate 150 is snapped into the position on the upper frame 120. This foot plate 150 can be snapped out for cleaning, and for this foot plate 150, this invention calls for use of a specific plastic material containing Phytoncides so that the foot plate 150 maintain antimicrobial ability at all times whether in use or not thereby suppressing build up of any micro organism while the apparatus is off. With this effective sanitary condition, combined with very high volume activity of anions plus UV light while the apparatus is on, the foot plate 150 becomes a highly sanitary place with strong ability to disinfect user's feet which is perhaps the body part most susceptible to microbial infection.

Referring to FIG. 3 and FIG. 4 and FIG. 5, the apparatus 10 with the foot plate 150 in place is turned on when a user's feet F stand on the foot plate 150 causing the weight of the user press down the weigh scale 160 which in turn switches on the apparatus 10. On the foot plate 150, there are curved grooves in a pattern propagating from the curved line of the passage opening 123 for aesthetics, but inside the grooves, there are the induction holes 151 whose inner walls are sloped in different angles specifically for the purpose of drawing the discharging air stream toward the body surface to optimize the coanda effect along the body surface, while the locations and sizes of the induction holes 151 to precisely match those of the nozzles 122a and 122b whose location and sizes are configured specifically to maximize the air stream near the user's body and increase the pressure of the air stream particularly at the user's groin area for thorough drying and maximum exposure to anions at the skin of user's body crevices.

When the apparatus 10 is turned on, blower fan 132 draws outside air into the blower unit 130 filtered through the air filter 138 and heated by the air heater 139, if so selected by the user, and push the air stream into the air chamber 111 sweeping the anions as they are discharged from the metal fiber brushes 141, and the air stream, cool or warm depending on the user selection and loaded with massive amount of anions, is discharged through the nozzles 122a and 122b sweeping the moisture off the user's skin while affording the skin to be in contact with very large amount of anions and let the user inhale large amount of anions, thereby affording the user a high level of health and beauty benefits of anion.

The temperature of the air heater 139 is adjusted by its own sensor based control circuitry to be turned on and off to maintain the air temperature inside the air chamber 111 at a selected level, thereby discharging air through the nozzles will be maintained at a preselected temperature.

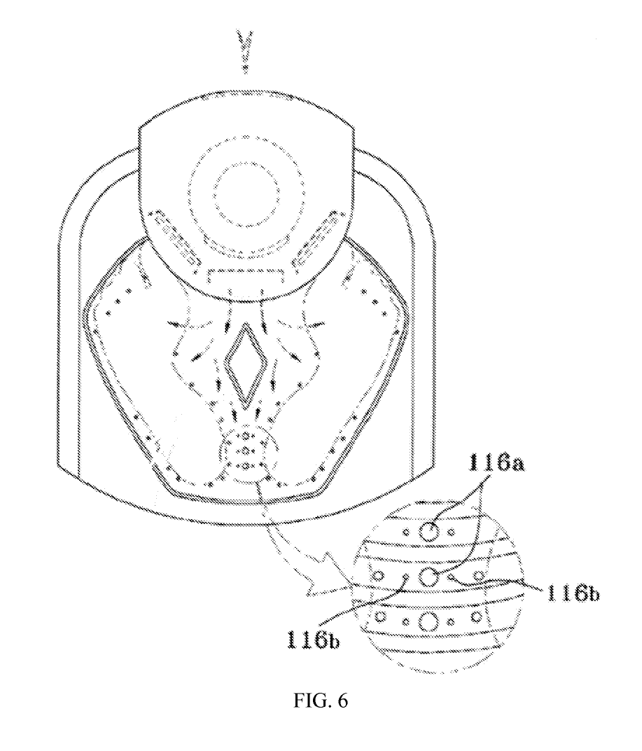

Referring to FIG. 6 and FIG. 7, above the air chamber 111 through its top cover 111b and through the top of the upper frame 120, there are many nozzles of large and small 122a and 122b neighboring each other with different discharge hole diameters, so that the discharged air stream from each meets the others from neighboring different size nozzles along the same upward flow direction gaining synergy effect in upward pressure, thereby reducing dispersion of the discharging air stream to slow down the rate of pressure drop of the upward air stream, resulting in increased travel height of the air stream at slower dispersion rate. In order to attain such synergy, the distance between the nozzle holes should not exceed 10 times of the nozzle outlet diameter.

For example, such upward air stream of high pressure owing to high CFM of the blower fan 132 combined with aforesaid air stream synergy effect, particularly at the larger nozzles 122a at the center, will reach the user's body crevices at the groin area at very high pressure thoroughly sweeping away residual moisture over, while pressing anions in the air stream strongly onto, the skin at such crevices, preventing microbial skin health hazards.

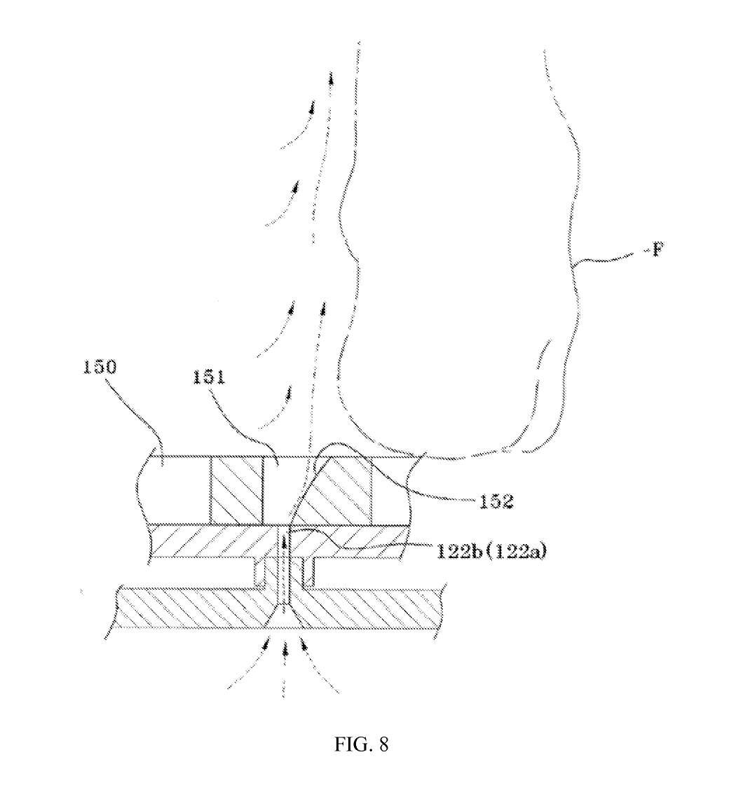

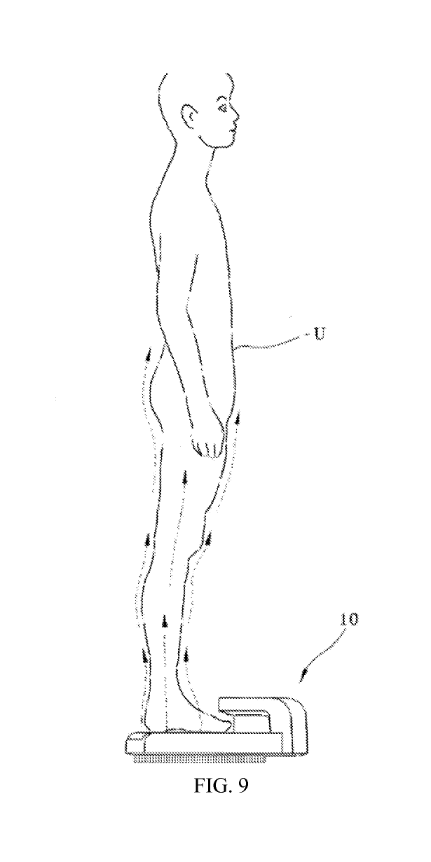

FIG. 8 and FIG. 9 further illustrate the induction holes 151 on the foot plate 150, whose wall angles are irregular with varying wall slope angles 152 that directs the discharging air stream to be induced toward the user's body along the body surface contour.

FIG. 10 illustrates the anion generator 140 of the type called for by this invention with its power supply 142, input power cords 146 and the anion generator leg 144 with metal fiber core of specific composition for the reason of keeping discharge of ozone which is the common byproduct of anion generation to a negligible level. Aforesaid metal fiber core is composed of an alloy of eight metals of platinum, stainless steel, copper, silver, zinc, nickel, manganese and tungsten, conductive cloth with free electrons, conductive gum and semi-conductive gum twisted like a rope and connected to the DC power supply 142 with the voltage level under 3 kv.

The apparatus 10 turns off by itself when the user steps down from the foot plate by the weighing scale stopping the on status and withdraw on signal to the weight display 161 and the main power switch of the apparatus 10 accordingly.

Although the invention has been explained in relation to its preferred embodiment, it is to be understood that many other possible modifications and variations can be made without departing from the spirit and scope of the invention as hereinafter described.

What is claimed:

1. An apparatus used to dry, weigh, as well as invigorate and sanitize the body comprises a lower frame, an upper frame, a blower unit, a sanitizing foot plate, and a weighing scale.

2. The apparatus used to dry, weigh, as well as invigorate and sanitize the body as claimed in claim 1, wherein the lower frame and the upper frame are positioned to properly support the blower unit, the sanitizing foot plate, the air chamber containing, and the weight scale.

3. The apparatus used to dry, weigh, as well as invigorate and sanitize the body as claimed in claim 1, wherein the blower unit is used to create a drying flow of air.

4. The apparatus used to dry, weigh, as well as invigorate and sanitize the body as claimed in claim 1, wherein the sanitizing foot plate is used to disinfect a user's feet and has design feature that optimizes coanda effect of the air stream.

5. The apparatus used to dry, weigh, as well as invigorate and sanitize the body as claimed in claim 1, wherein the anion generators are used to generate anions and mix them into the air stream to be in contact with the body of and inhale by the user for health benefits.

6. The apparatus used to dry, weigh, as well as invigorate and sanitize the body as claimed in claim 1, wherein the weighing scale is used to measure a user's weight.
